(19) United States
(12) Patent Application Publication
(10) Pub. No.: US 2021/0155611 A1
(43) Pub. Date: May 27, 2021

(54) THE SALTS OF A COMPOUND AND THE CRYSTALLINE FORMS THEREOF

(71) Applicant: Flutchison Medipharma Limited, Shanghai (CN)

(72) Inventors: Zhenping Wu, Shanghai (CN); Bo Liu, Shanghai (CN); Weniji Li, Shanghai (CN); Yuping Chu, Shanghai (CN); Ling Feng, Shanghai (CN); Zhixiang Shen, Shanghai (CN)

(21) Appl. No.: 16/614,029

(22) PCT Filed: May 16, 2018
(86) PCT No.: PCT/CN2018/087047

§ 371 Date: Nov. 15, 2019

(30) Foreign Application Priority Data
May 16, 2017 (CN) 201710343882.5

(51) Int. Cl. C07D 403/12

(52) U.S. Cl. CPC C07D 403/12

(57) ABSTRACT

The present invention belongs to the pharmaceutical field, and provides the compound 4-ethyl-N-(4-((3-ethynyiphenypamino)-7-methoxyquinazolin-6-yDpiperazine-1-carboxamide succinate and the crystalline forms thereof, the solvates and the crystalline forms thereof, the pharmaceutical compositions comprising the same as well as the methods of preparing the same and the use thereof.

### FIELD OF THE INVENTION

The present invention belongs to the pharmaceutical field, and provides the compound 4-ethyl-N-(4-((3-ethynylphenyl)amino)-7-methoxyquinazolin-6-yl)piperazine-1-carboxamide succinate and the crystalline forms thereof, the solvates and the crystalline forms thereof, the pharmaceutical compositions comprising the same as well as the methods of preparing the same and the use thereof.

### BACKGROUND OF THE INVENTION

Binding of epidermal growth factor (EGF) to epidermal growth factor receptor (EGFR) activates tyrosine kinase activity and thereby triggers reactions that lead to cellular proliferation. Overexpression and/or overactivity of EGFR could result in uncontrolled cell division which may be a predisposition for cancer. Compounds that inhibit the overexpression and/or overactivity of EGFR are therefore candidates for treating cancer.

The relevant compound 4-ethyl-N-(4-((3-ethynylphenyl)amino)-7-methoxyquinazolin-6-yl) piperazine-1-carboxamide of the present invention has the effect of effectively inhibiting the overexpression and/or overactivity of EGFR. Thus, it is useful in treating diseases associated with overexpression and/or overactivity of EGFR, such as the treatment of cancer.

The phenomenon that a compound could exist in two or more crystal structures is known as polymorphism. Many compounds may exist as various polymorph crystals and also in a solid amorphous form. Until polymorphism of a compound is discovered, it is highly unpredictable (1) whether a particular compound will exhibit polymorphism, (2) how to prepare any such unknown polymorphs, and (3) how are the properties, such as stability, of any such unknown polymorphs. See, e.g., J. Bernstein “Polymorphism in Molecular Crystals”, Oxford University Press, (2002).

Since the properties of a solid material depend on the structure as well as on the nature of the compound itself, different solid forms of a compound can and often do exhibit different physical and chemical properties as well as different biopharmaceutical properties. Differences in chemical properties can be determined, analyzed and compared through a variety of analytical techniques. Those differences may ultimately be used to differentiate among different solid forms. Furthermore, differences in physical properties, such as solubility, and biopharmaceutical properties, such as bioavailability, are also of importance when describing the solid state of a pharmaceutical compound. Similarly, in the development of a pharmaceutical compound, e.g., 4-ethyl-N-(4-((3-ethynylphenyl)amino)-7-methoxyquinazolin-6-yl)piperazine-1-carboxamide, the new crystalline and amorphous forms of the pharmaceutical compound are also of importance.

The compound 4-ethyl-N-(4-((3-ethynylphenyl)amino)-7-methoxyquinazolin-6-yl) piperazine-1-carboxamide as well as the preparation thereof was described in patent CN101619043A.

### CONTENTS OF THE INVENTION

### Summary

Upon extensive explorations and research, we have found that compound 4-ethyl-N-(4-((3-ethynylphenyl)amino)-7-methoxyquinazolin-6-yl)piperazine-1-carboxamide can be prepared into succinate salts, the chemical structure of its semisuccinate and monosuccinate being shown by Formula A. Studies have shown that, compared with its free base, the solubility of compound of Formula A is significantly increased, which is beneficial for improving the pharmacokinetic characteristics and in vivo bioavailability of the compound. We have also found that compound of Formula A can exist in different crystalline forms, and can form solvates with certain solvents. We have made extensive studies on the polymorphic forms of compound of Formula A and have finally prepared and determined the polymorphic forms which meet the requirement of pharmaceutical use. Based on these studies, the present invention provides the compound 4-ethyl-N-(4-((3-ethynylphenyl)amino)-7-methoxyquinazolin-6-yl)piperazine-1-carboxamide succinate and the various crystalline forms thereof, solvates and the crystalline forms thereof, which are designated as Form I, Form IV and Form V respectively.

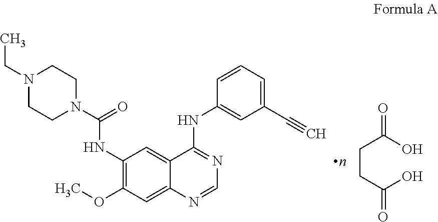

wherein, n is 0.5 or 1.

In one aspect, the polymorphs forms of compound of Formula A or the solvates thereof provided by the present invention have good crystallinity, non-hygroscopicity, and good stability.

Firstly, the present invention provides the succinate salts of 4-ethyl-N-(4-((3-ethynylphenyl) amino)-7-methoxyquinazolin-6-yl)piperazine-1-carboxamide.

Secondly, the present invention provides 4-ethyl-N-(4-((3-ethynylphenyl)amino)-7-methoxy quinazolin-6-yl)piperazine-1-carboxamide succinate of Formula A, i.e. compound of Formula A.

Further, the present invention provides crystalline Form I of 4-ethyl-N-(4-((3-ethynylphenyl) amino)-7-methoxyquinazolin-6-yl)piperazine-1-carboxamide succinate, i.e. Form I of compound of Formula A (wherein, n is 0.5).

Even further, the present invention provides the solvates of 4-ethyl-N-(4-((3-ethynylphenyl) amino)-7-methoxyquinazolin-6-yl)piperazine-1-carboxamide succinate, which may be hydrate, and water and acetone solvate of compound of Formula A (wherein, n is 0.5).

Even further, the present invention provides the solvates of 4-ethyl-N-(4-((3-ethynylphenyl) amino)-7-methoxyquinazolin-6-yl)piperazine-1-carboxamide succinate, which may be dihydrate, and water and acetone solvate (containing 1.5 molecules of water and 1 molecule of acetone) of compound of Formula A (wherein, n is 0.5).

Even further, the present invention provides the solvates of 4-ethyl-N-(4-((3-ethynylphenyl) amino)-7-methoxyquinazolin-6-yl)piperazine-1-carboxamide succinate, which may be hydrate, and the water and acetonitrile solvate of compound of Formula A (wherein, n is 1).

Even further, the present invention provides the solvates of 4-ethyl-N-(4-((3-ethynylphenyl) amino)-7-methoxyquinazolin-6-yl)piperazine-1-carboxamide succinate, which may be hemihydrate, and the water and acetonitrile solvate (containing 2.5 molecules of water and 0.3 molecule of acetonitrile) of compound of Formula A (wherein, n is 1).

Even further, the present invention provides the water and acetonitrile solvate of 4-ethyl-N-(4-((3-ethynylphenyl)amino)-7-methoxyquinazolin-6-yl)piperazine-1-carboxamide succinate, which may be the water and acetonitrile solvate (containing 2.5 molecules of water and 0.3 molecule of acetonitrile) of compound of Formula A (wherein, n is 1), which may be Form IV.

Even further, the present invention provides hemihydrate of 4-ethyl-N-(4-((3-ethynylphenyl) amino)-7-methoxyquinazolin-6-yl)piperazine-1-carboxamide succinate, which may be hemihydrate of compound of Formula A (wherein, n is 1), which may be Form V.

In another aspect, the present invention provides the methods of preparation for compound of Formula A or its solvates or the crystalline forms of compound of Formula A or its solvates (such as Form 1, Form IV, and Form V), which are reproducible and easy in operation.

In still another aspect, the present invention provides the pharmaceutical compositions comprising an effective amount of one or more of compound of Formula A or its solvates or the crystalline forms of compound of Formula A or its solvates (such as Forms I, Form IV, and Form V), and remainder amount of at least one pharmaceutically acceptable carrier.

The present invention further provides a method of treating cancer responsive to inhibition of overexpression and/or overactivity of epidermal growth factor receptor, comprising administering to a subject in need thereof an effective amount of one or more of compound of Formula A or its solvates or the crystalline forms of compound of Formula A or its solvates of the present invention, such as Forms I, Form IV, or Form V.

The present invention further provides a use of compound of Formula A or its solvates or the crystalline forms of compound of Formula A or its solvates (such as Forms I, Form IV, or Form V) in the manufacture of a medicament for treating cancer responsive to inhibition of overexpression and/or overactivity of epidermal growth factor receptor, such as lung cancer (including non-small cell lung cancer, non-small cell lung cancer with brain metastasis), head and neck cancer, (large) intestinal cancer, colorectal cancer, rectal cancer, colon cancer, pancreatic cancer, brain cancer (including glioblastoma), breast cancer, pharynx cancer, epidermoid cancer, ovarian cancer, prostate cancer, gastric cancer, renal cancer, liver cancer, esophageal cancer, bone cancer, sarcoma such as soft tissue sarcoma, and leukemia.

### BRIEF DESCRIPTION OF THE FIGURES

FIG. 1 shows a X-ray powder diffractogram of Form I of compound of Formula A, wherein the horizontal axis (X-axis) plots the diffraction 2 theta, and the vertical axis (Y-axis) plots the diffraction intensity (%).

FIG. 2 shows a differential scanning calorimetry (DSC) profile of Form I of compound of Formula A, wherein the horizontal axis (X-axis) plots the temperature (° C.), and the vertical axis (Y-axis) plots the heat flow (mW).

FIG. 3 shows a Thermogravimetric (TG) profile of Form I of compound of Formula A, wherein the horizontal axis (X-axis) plots the temperature (° C.), and the vertical axis (Y-axis) plots the weight percentage (%).

FIG. 4 shows a X-ray powder diffractogram of Form IV of the water and acetonitrile solvate of compound of Formula A, wherein the horizontal axis (X-axis) plots the diffraction 2 theta, and the vertical axis (Y-axis) plots the diffraction intensity (%).

FIG. 5 shows a differential scanning calorimetry (DSC) profile of Form IV of the water and acetonitrile solvate of compound of Formula A, wherein the horizontal axis (X-axis) plots the temperature (° C.), and the vertical axis (Y-axis) plots the heat flow (mW).

FIG. 6 shows a Thermogravimetric (TG) profile of Form 1V of the water and acetonitrile solvate of compound of Formula A, wherein the horizontal axis (X-axis) plots the temperature (° C.), and the vertical axis (Y-axis) plots the weight percentage (%).

FIG. 7 shows a X-ray powder diffractogram of Form V of hemihydrate of compound of Formula A, wherein the horizontal axis (X-axis) plots the diffraction 2 theta, and the vertical axis (Y-axis) plots the diffraction intensity (%).

FIG. 8 shows a differential scanning calorimetry (DSC) profile of Form V of hemihydrate of compound of Formula A, wherein the horizontal axis (X-axis) plots the temperature (° C.), and the vertical axis (Y-axis) plots the heat flow (mW).

FIG. 9 shows a Thermogravimetric (TG) profile of Form V of hemihydrate of compound of Formula A, wherein the horizontal axis (X-axis) plots the temperature (° C.), and the vertical axis (Y-axis) plots the weight percentage (%).

### DEFINITIONS

Unless indicated otherwise, the following abbreviations or terms as used in the present application (including the specification and the claims) have the meanings as set forth below. It is to be noted that the singular forms “a”, “an” and “the” in the specification and the claims include plural referents, unless clearly indicated otherwise.

The term “crystalline forms of the present invention” as used herein refers to crystalline forms Form I, Form IV or Form V of compound of Formula A or the solvates thereof, or a mixture thereof. “Form”, “crystalline form” and “polymorph” may be used interchangeably herein.

The term “compound of Formula A” as used herein refers to a compound having the following chemical structure of Formula A (also referenced as “Compound A”):

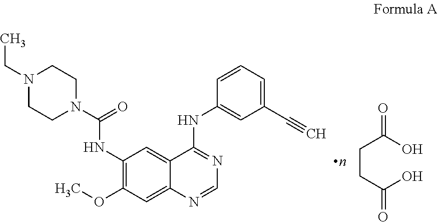

wherein, n is 0.5 or 1.

The term “C₁₋₆ alkanol” as used herein refers to a fully saturated straight or branched alkyl alcohol having 1, 2, 3, 4, 5 or 6 carbon atoms. Examples include but not limited to methanol, ethanol, n-propanol, i-propanol, n-butanol, i-butanol, t-butanol, n-pentanol, i-pentanol, n-hexanol, and the like.

The term “haloalkane with less than three carbon atoms” as used herein refers to fully saturated hydrocarbon having 1 or 2 carbon atoms, which is substituted with one or more halogen atoms selected from F, Cl, Br or I. Examples include dichloromethane, trichloromethane, carbon tetrachloride, 1,2-dichloroethane, and the like.

The term “about” as used herein refers to the deviation from a given numerical value of no more than 10%.

The term “substantially free of other forms” as used herein means that the content of said other forms is less than 50%, preferably less than 40%, preferably less than 30%, preferably less than 20%, preferably less than 10%, preferably less than 5%, preferably less than 1% by weight, based on the total weight of the forms.

The term “solution” as used herein means a mixture of one or more solutes in one or more solvents, for certain use. Solution is intended to encompass homogeneous mixtures as well as heterogeneous mixtures, such as slurries or other suspension mixtures having insoluble (not dissolved) material.

The term “organic solvent” as used herein is broadly intended to mean any appropriate organic solvent for certain use disclosed herein.

The term “dissolution solvent” as used herein refers to any appropriate organic solvent which is capable of dissolving, in whole or in part, the solutes under appropriate conditions, such as an appropriate amount and an appropriate temperature, such as room temperature or an elevated temperature.

The term “anti-dissolution solvent” as used herein refers to any appropriate organic solvent in which the substrate has less solubility than in the dissolution solvent.

The term “effective amount” of compound of Formula A and the crystalline forms thereof, solvates and the crystalline forms thereof means an amount which is effective in alleviating, improving, or stopping or delaying the progression of cancer responsive to inhibition of overexpression and/or overactivity of epidermal growth factor receptor when administered to an individual, which may be a human, animal or the like, wherein the cancer responsive to inhibition of overexpression and/or overactivity of epidermal growth factor receptor includes but not limited to lung cancer (including non-small cell lung cancer, non-small cell lung cancer with brain metastasis), head and neck cancer, (large) intestinal cancer, colorectal cancer, rectal cancer, colon cancer, pancreatic cancer, brain cancer (including glioblastoma), breast cancer, pharynx cancer, epidermoid cancer, ovarian cancer, prostate cancer, gastric cancer, renal cancer, liver cancer, esophageal cancer, bone cancer, sarcoma such as soft tissue sarcoma, and leukemia. “Effective amount” may vary with various factors, such as compound, state of disease to be treated, severity of disease to be treated, age and health status of the individual, administration route and form, judgement of the attending physician or a veterinary practitioner, and so on.

The term “individual” or “subject” as used herein means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but not limited to, birds, and the like. The term “individual” or “subject” does not denote a particular age or sex.

### DETAILED DESCRIPTION OF THE INVENTION

The present invention provides compound 4-ethyl-N-(4-((3-ethynylphenyl)amino)-7-methoxyquinazolin-6-yl)piperazine-1-carboxamide succinate and the crystalline forms thereof, solvates and the crystalline forms thereof.

The crystalline forms of the present invention have good crystallinity, non-hygroscopicity, and good stability. The crystalline forms of the present invention have good reproducibility and can realize repeatable amplified production; moreover, they are stable in ordinary formulations, so it is convenient for using in the manufacture of formulations and treatment of diseases. In addition, the crystalline forms of the present invention have high purity and less solvent residue, which meet the quality requirements of bulk drug, such as ICH Q3A.

The person of ordinary skill in the art can verify the above advantages of the crystalline forms of the present invention according to the test methods disclosed in the pharmacopoeias and the modification thereof, or the conventional methods in the art.

As described herein, the crystalline forms of the present invention may be identified by one or more solid state analytical methods. For example, the crystalline forms of the present invention may be identified by one or more methods, e.g., X-ray powder diffraction, lattice parameters of a single crystal, Fourier Infrared Spectroscopy, differential scanning calorimetry analytical data, and/or a thermogravimetric curve. Moreover, if the identified or analytical result by one of those methods is consistent with that of the forms of the present invention, it does not mean that the result by any other methods is consistent with that of the forms of the present invention.

As described herein, the new crystalline forms may be identified according to X-ray powder diffraction. However, it is known in the art that the peak intensity and/or measured peaks in the X-ray powder diffractogram may vary with the different experiment condition, e.g., different diffraction test conditions and/or preferred orientations or like. Furthermore, the observed 2θ value may be subjected to an error of about ±0.2 2θ due to different instrument precision. However, it is known that, compared with the positions of peaks, the relative intensity values of the peaks more depend on certain properties of the tested samples, e.g., crystal size in the sample, orientation effect of crystalline and purity of the analysed materials. Therefore, the deviation of the peak intensity at about ±20% or greater may occur. However, despite of experimental errors, machine errors, preferred orientation and the like, one skilled in the art can obtain sufficient information from the XRPD data provided herein to identify Form I and any other crystalline forms of the present invention.

### Form I

The present invention provides Form 1 of compound of Formula A (wherein, n is 0.5).

In some embodiments, Form I of compound of Formula A may be identified according to X-ray powder diffraction. In some embodiments, the X-ray powder diffraction characteristic diffraction angles (2θ) of Form I of compound of Formula A include 6.1, 7.9, 12.2, 15.3, 15.9, 16.6, and 20.4 degrees, the measured 2θ values each having an error of about ±0.2 degrees (2θ).

In some embodiments, the X-ray powder diffraction characteristic diffraction angles (2θ) of Form I of compound of Formula A include 6.1, 7.9, 12.2, 15.3, 15.9, 16.6, 20.4, and 21.7 degrees, the measured 2θ values each having an error of about ±0.2 degrees (2θ).

In some embodiments, the X-ray powder diffraction characteristic diffraction angles (2θ) of Form I of compound of Formula A include 6.1, 7.9, 10.2, 11.6, 12.2, 15.3, 15.9, 16.6, 17.8, 19.6, 20.4, 21.7, and 23.5 degrees, the measured 2θ values each having an error of about ±0.2 degrees (2θ).

In some embodiments, the X-ray powder diffraction characteristic diffraction angles (2θ) of Form I of compound of Formula A include 6.1, 7.9, 10.2, 11.6, 12.2, 13.6, 15.3, 15.9, 16.6, 17.8, 18.3, 19.6, 20.4, 21.4, 21.7, 22.3, 23.5, 24.3, and 25.1 degrees, the measured 2θ values each having an error of about ±0.2 degrees (2θ).

In some embodiments, the X-ray powder diffraction characteristic diffraction angles (2θ) of Form I of compound of Formula A include 6.1, 7.9, 10.2, 11.6, 12.2, 13.2, 13.6, 14.6, 15.3, 15.9, 16.6, 17.8, 18.3, 19.6, 20.4, 21.4, 21.7, 22.3, 23.5, 24.3, 25.1, 26.2, 26.9, 27.5, and 28.2 degrees, the measured 2θ values each having an error of about ±0.2 degrees (2θ).

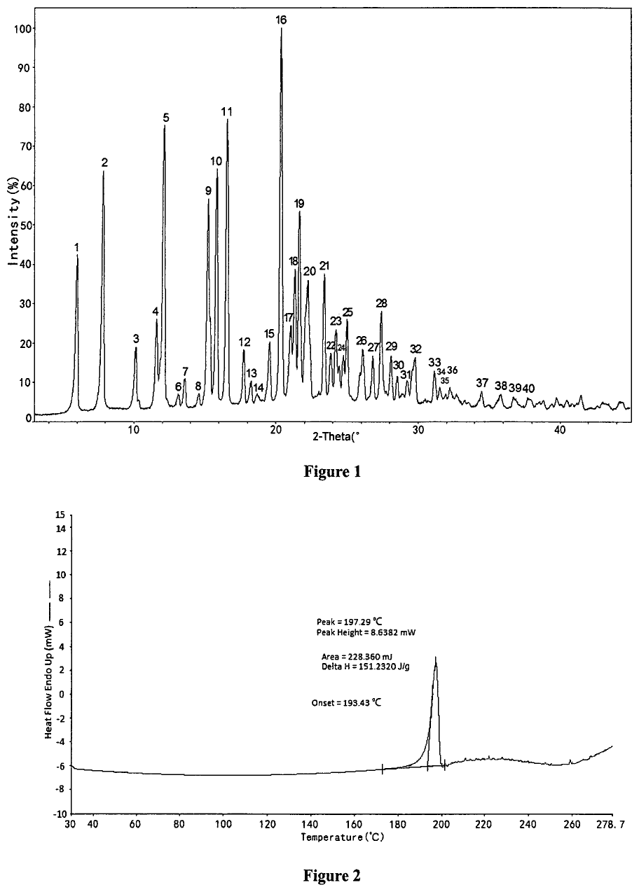

In some embodiments, Form I of compound of Formula A has a diffractogram as shown in FIG. 1. Despite of experimental errors, machine errors, preferred orientation and the like, one skilled in the art can obtain sufficient information from the XRPD data provided herein to identify Form I of compound of Formula A.

In some embodiments, Form I of compound of Formula A may be characterized by differential scanning calorimetry (DSC). In some embodiments, Form I of compound of Formula A has a DSC curve as shown in FIG. 2. In the DSC profile, the endothermic peak of Form I of compound of Formula A is at about 193.4-197.3° C.

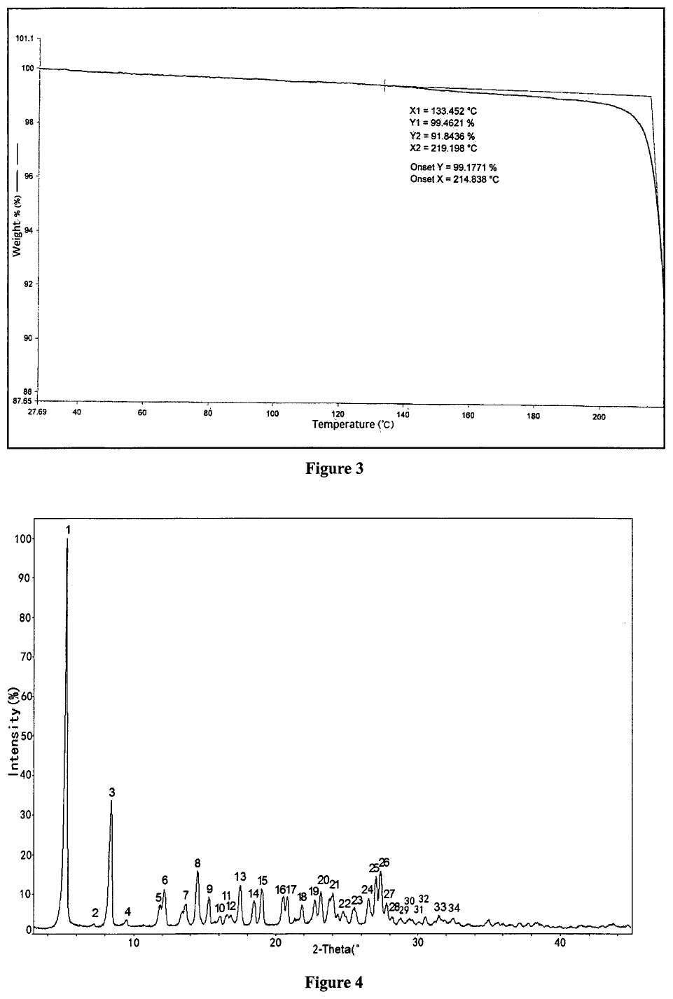

In some embodiments, Form I of compound of Formula A may be characterized by thermogravimetric analysis (TGA). In some embodiments, Form I of compound of Formula A has a TGA curve as shown in FIG. 3, indicating that Form I is an anhydrous material or a neat crystal.

In some embodiments, Form I of compound of Formula A is substantially free of other crystalline forms as described herein. For example, the content by weight of Form I of compound of Formula A is at least 99%, at least 95%, at least 90%, or even lower to 80%. Further, the content by weight of Form I of compound of Formula A is at least 70%, or at least 60%. Even further, the content by weight of Form I of compound of Formula A is at least 50%.

Methods of Preparing Form I Method A The present invention relates to a method of preparing Form I of compound of Formula A, comprising:

(1) mixing the compound 4-ethyl-N-(4-((3-ethynylphenyl)amino)-7-methoxyquinazolin-6-yl)piperazine-1-carboxamide with succinic acid in an appropriate amount of at least one dissolution solvent or of a mixed solvent consisting of water miscible organic solvent and water, heating and stirring to form a salt;

(2) naturally cooling the reaction solution obtained in step (1) to room temperature with stirring;

(3) isolating to obtain the solid Form I of 4-ethyl-N-(4-((3-ethynylphenyl)amino)-7-methoxyquinazolin-6-yl)piperazine-1-carboxamide succinate;

(4) drying the solid obtained in step (3).

In some embodiments, the mole ratio of succinic acid to compound 4-ethyl-N-(4-((3-ethynylphenyl)amino)-7-methoxyquinazolin-6-yl)piperazine-1-carboxamide is no less than about 1:1. In some embodiments, the mole ratio is about 2.5:1. In some embodiments, the mole ratio is about 4:1.

In some embodiments, the ratio of the volume (mL) of the dissolution solvent or the mixed solvent to the weight (g) of compound 4-ethyl-N-(4-((3-ethynylphenyl) amino)-7-methoxyquinazolin-6-yl)piperazine-1-carboxamide in step (1) is no less than about 10 mL/g (volume/weight ratio), such as 10 mL/g, 15 mL/g, 150 mL/g.

In some embodiments, the dissolution solvent is selected from C_{f} alkanol, tetrahydrofuran, haloalkane with less than three carbon atoms, acetone, butanone, and acetonitrile. In some embodiments, the dissolution solvent is selected from ethanol and tetrahydrofuran.

In some embodiments, the volume percentage of said water miscible organic solvent in said mixed solvent is less than about 95%.

In some embodiments, said water miscible organic solvent is selected from acetone, methanol, ethanol, i-propanol, tetrahydrofuran, and acetonitrile. In some embodiments, said water miscible organic solvent is selected from ethanol, and the volume percentage of ethanol in said mixed solvent is no less than about 50%.

In some embodiments, said water miscible organic solvent and water are mixed in an appropriate ratio. In some embodiments, the volume ratio of the water miscible organic solvent to water is about 13:1, such as ethanol/water (about 13:1 in V/V).

In some embodiments, the heating temperature in step (1) is not higher than the boiling point of the solvent system, such as about 50° C., about 60° C., and about 80° C.

In some embodiments, the stirring time in step (2) may range from 12 to 100 hours, such as at least 12 hours, at least 18 hours, at least 24 hours, at least 48 hours.

In some embodiments, the drying temperature and drying time in step (4) should be appropriate so that the solid is dried sufficiently and the desired crystalline properties are maintained. In some embodiments, the drying temperature is 40° C.

Method B The present invention provides a further method of preparing Form I of compound of Formula A, comprising:

(1) adding the compound 4-ethyl-N-(4-((3-ethynylphenyl)amino)-7-methoxyquinazolin-6-yl) piperazine-1-carboxamide and succinic acid into an appropriate amount of at least one dissolution solvent or of a mixed solvent consisting of water miscible organic solvent and water, and reacting to form a salt, thereby obtaining the first solution;

(2) adding at least one anti-dissolution solvent into said first solution to obtain the second solution;

(3) isolating to obtain the solid Form I of 4-ethyl-N-(4-((3-ethynylphenyl)amino)-7-methoxyquinazolin-6-yl)piperazine-1-carboxamide succinate;

(4) drying the solid obtained in step (3).

In some embodiments, said dissolution solvent is selected from one or more of methanol and dichloromethane.

In some embodiments, said water miscible organic solvent is selected from acetone, and i-propanol.

In some embodiments, said water miscible organic solvent and water are mixed in an appropriate ratio. In some embodiments, the volume ratio of the water miscible organic solvent to water ranges from about 25:1 to 3:1, such as i-propanol/water (about 8.3:1 in V/V), acetone/water (about 3.3:1 in V/V).

In some embodiments, said anti-dissolution solvent is selected from ethyl acetate, acetone, and i-propanol.

In some embodiments, the volume ratio of the dissolution solvent or the mixed solvent to the anti-dissolution solvent ranges from about 1:2 to about 2:1, such as 1:1, 1:2, 1.3:1.

In some embodiments, both stirring and heating are applied in said step (1). The heating temperature is not higher than the boiling point of the solvent system, such as about 40° C., about 60° C., and about 80° C.

### Water and Acetonitrile Solvate

The present invention further provides the water and acetonitrile solvate of compound of Formula A (wherein, n is 1).

In some embodiments, the water and acetonitrile solvate of compound of Formula A (wherein, n is 1) contains 2.5 molecules of water and 0.3 molecule of acetonitrile.

In some embodiments, the water and acetonitrile solvate (containing 2.5 molecules of water and 0.3 molecule of acetonitrile) of compound of Formula A (wherein, n is 1) is Form IV.

In some embodiments, Form IV of the water and acetonitrile solvate of compound of Formula A may be characterized through X-ray powder diffraction. The X-ray powder diffraction characteristic diffraction angles (2θ) of Form IV include 5.4, 8.5, 12.2, 14.5, and 15.3 degrees, the measured 2θ values each having an error of about ±0.2 degrees (2θ).

In some embodiments, the X-ray powder diffraction characteristic diffraction angles (2θ) of Form IV of the water and acetonitrile solvate of compound of Formula A include 5.4, 8.5, 9.6, 12.2, 14.5, 15.3, 17.5, 19.1, 22.8, and 27.1 degrees, the measured 2θ values each having an error of about ±0.2 degrees (2θ).

In some embodiments, the X-ray powder diffraction characteristic diffraction angles (2θ) of Form IV of the water and acetonitrile solvate of compound of Formula A include 5.4, 7.3, 8.5, 9.6, 11.9, 12.2, 13.7, 14.5, 15.3, 17.5, 18.5, 19.1, 20.6, 22.8, and 27.1 degrees, the measured 2θ values each having an error of about +0.2 degrees (2θ).

In some embodiments, the X-ray powder diffraction characteristic diffraction angles (2θ) of Form IV of the water and acetonitrile solvate of compound of Formula A include 5.4, 7.3, 8.5, 9.6, 11.9, 12.2, 13.7, 14.5, 15.3, 17.5, 18.5, 19.1, 20.6, 20.9, 21.9, 22.8, 24.1, 26.6, 27.1, 27.4, and 27.8 degrees, the measured 2θ values each having an error of about +0.2 degrees (2θ).

In some embodiments, Form IV of the water and acetonitrile solvate of compound of Formula A has a diffractogram as shown in FIG. 4. Despite of experimental errors, machine errors, preferred orientation and the like, one skilled in the art can obtain sufficient information from the XRPD data provided herein to identify Form IV of the water and acetonitrile solvate of compound of Formula A.

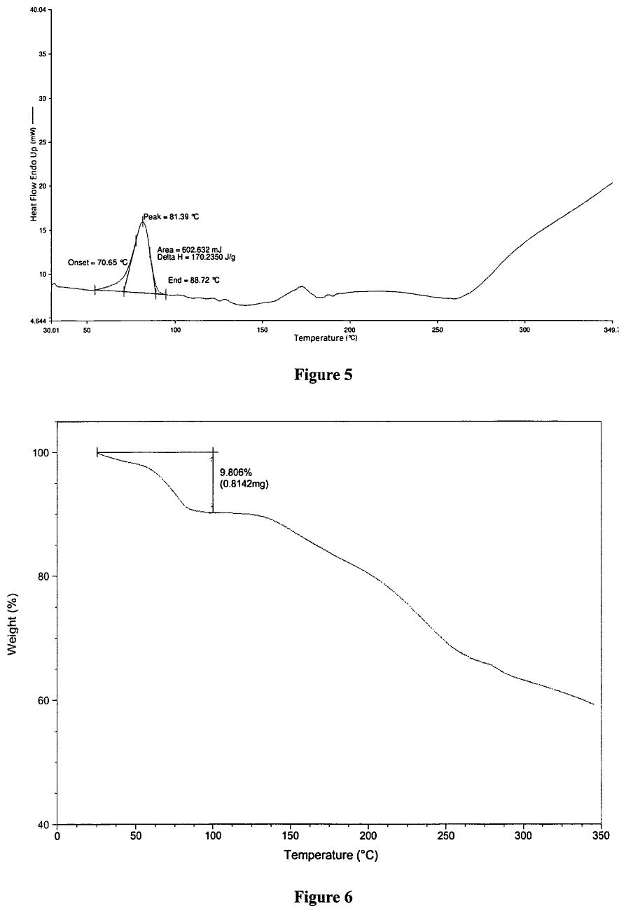

In some embodiments, Form IV of the water and acetonitrile solvate of compound of Formula A may be characterized by differential scanning calorimetry (DSC). In some embodiments, Form IV of the water and acetonitrile solvate of compound of Formula A has a DSC curve as shown in FIG. 5. In the DSC profile, the endothermic peak of Form IV of the water and acetonitrile solvate of compound of Formula A is at about 70.6-81.4° C.

In some embodiments, Form IV of the water and acetonitrile solvate of compound of Formula A may be characterized by thermogravimetric analysis (TGA). In some embodiments, Form IV of the water and acetonitrile solvate of compound of Formula A has a TGA curve as shown in FIG. 6, indicating that Form IV is a water and acetonitrile solvate (containing 2.5 molecules of water and 0.3 molecule of acetonitrile).

In some embodiments, Form IV of the water and acetonitrile solvate of compound of Formula A is substantially free of other crystalline forms as described herein. For example, the content by weight of Form IV of the water and acetonitrile solvate of compound of Formula A is at least 99%, at least 95%, at least 90%, or even lower to 80%. Further, the content by weight of Form IV of the water and acetonitrile solvate of compound of Formula A is at least 70%, or at least 60%. Even further, the content by weight of Form IV of the water and acetonitrile solvate of compound of Formula A is at least 50%.

### Methods of Preparing Form IV

The present invention provides a method of preparing Form IV of the water and acetonitrile solvate of compound of Formula A, comprising:

(1) adding the compound 4-ethyl-N-(4-((3-ethynylphenyl)amino)-7-methoxyquinazolin-6-yl) piperazine-1-carboxamide and succinic acid into an appropriate amount of a mixed solvent of acetonitrile and water and reacting to form a salt, thereby obtaining a clear solution;

(2) adding acetonitrile to the solution obtained in step (1);

(3) stirring the suspension obtained in step (2);

(4) isolating to obtain the solid Form IV of 4-ethyl-N-(4-((3-ethynylphenyl)amino)-7-methoxyquinazolin-6-yl)piperazine-1-carboxamide succinate;

(5) drying the solid obtained in step (4).

In some embodiments, the mole ratio of said succinic acid to compound 4-ethyl-N-(4-((3-ethynylphenyl)amino)-7-methoxyquinazolin-6-yl)piperazine-1-carboxamide is no less than about 2:1. In some embodiments, the mole ratio is about 2:1. In some embodiments, the mole ratio is about 2.5:1.

In some embodiments, the volume ratio of acetonitrile to water used in step (1) is about 2:1.

In some embodiments, the ratio of the volume (mL) of said acetonitrile used in step (2) to the weight (g) of compound 4-ethyl-N-(4-((3-ethynylphenyl)amino)-7-methoxyquinazolin-6-yl) piperazine-1-carboxamide is no less than about 20 mL/g (volume/weight ratio).

As described herein, the stirring temperature is lower than the boiling point of the solvent system, such as room temperature, and about 40° C.

In some embodiments, the time of stirring the suspension in step (3) may range from 12 to 100 hours, such as at least 12 hours, at least 24 hours, at least 48 hours, at least 60 hours.

In some embodiments, the drying temperature and drying time in step (5) should be appropriate so that the solid is dried sufficiently and the desired crystalline properties are maintained.

In some embodiments, in step (5), the drying temperature is room temperature, and the drying time is 4 hours.

### Hemihydrate

The present invention further provides hydrate of compound of Formula A (wherein, n is 1).

In some embodiments, hydrate of compound of Formula A (wherein, n is 1) is hemihydrate.

In some embodiments, hemihydrate of compound of Formula A (wherein, n is 1) is Form V.

In some embodiments, Form V of hemihydrate of compound of Formula A may be characterized by X-ray powder diffraction. The X-ray powder diffraction characteristic diffraction angles (2θ) of Form V include 6.0, 8.9, 9.6, and 14.5 degrees, the measured 2θ values each having an error of about ±0.2 degrees (2θ).

In some embodiments, the X-ray powder diffraction characteristic diffraction angles (2θ) of Form V of hemihydrate of compound of Formula A include 6.0, 8.9, 9.6, 14.5, 19.2, 20.2, and 22.5 degrees, the measured 2θ values each having an error of about ±0.2 degrees (2θ).

In some embodiments, the X-ray powder diffraction characteristic diffraction angles (2θ) of Form V of hemihydrate of compound of Formula A include 6.0, 8.9, 9.6, 12.0, 14.5, 16.2, 17.5, 19.2, 20.2, 22.5, and 27.8 degrees, the measured 2θ values each having an error of about ±0.2 degrees (2θ).

In some embodiments, the X-ray powder diffraction characteristic diffraction angles (2θ) of Form V of hemihydrate of compound of Formula A include 6.0, 8.9, 9.6, 12.0, 14.5, 15.6, 16.2, 17.5, 19.2, 20.2, 22.5, 23.9, 27.2, and 27.8 degrees, the measured 2θ values each having an error of about ±0.2 degrees (2θ).

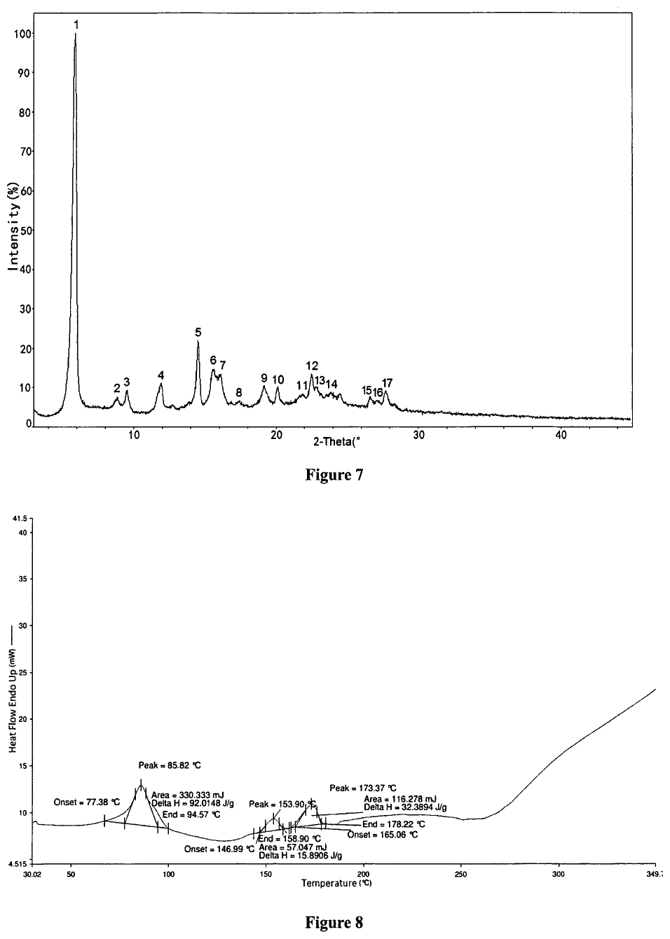

In some embodiments, Form V of hemihydrate of compound of Formula A has a diffractogram as shown in FIG. 7. Despite of experimental errors, machine errors, preferred orientation and the like, one skilled in the art can obtain sufficient information from the XRPD data provided herein to identify Form V of hemihydrate of compound of Formula A.

In some embodiments, Form V of hemihydrate of compound of Formula A may be characterized by differential scanning calorimetry (DSC). In some embodiments, Form V of hemihydrate of compound of Formula A has a DSC curve as shown in FIG. 8. In the DSC thermogram, the endothermic peaks of Form V of hemihydrate of compound of Formula A are at about 77.4-85.8° C., about 147.0-153.9° C., and about 165.1-173.4° C.

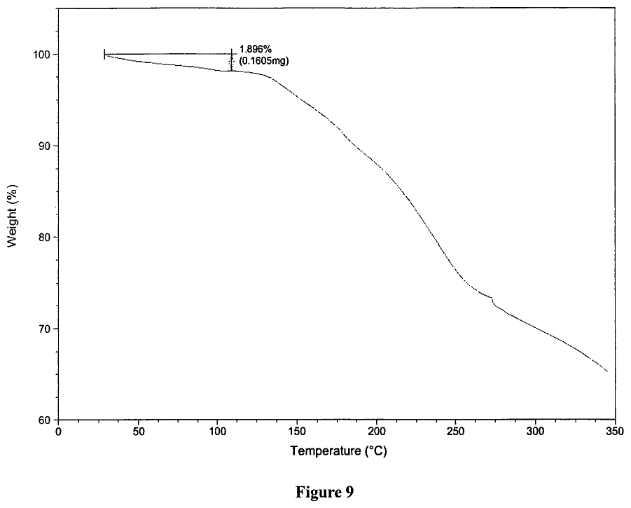

In some embodiments, Form V of hemihydrate of compound of Formula A may be characterized by thermogravimetric analysis (TGA). In some embodiments, Form V of hemihydrate of compound of Formula A has a TGA curve as shown in FIG. 9, indicating that Form V is a hemihydrate.

In some embodiments, Form V of hemihydrate of compound of Formula A is substantially free of other crystalline forms as described herein. For example, the content by weight of Form V of hemihydrate of compound of Formula A is at least 99%, at least 95%, at least 90%, or even lower to 80%. Further, the content by weight of Form V of hemihydrate of compound of Formula A is at least 70%, or at least 60%. Even further, the content by weight of Form V of hemihydrate of compound of Formula A is at least 50%.

Methods of Preparing Form V The present invention provides a method of preparing Form V of hemihydrate of compound of Formula A, comprising: drying the sample of Form IV of compound of Formula A at 60° C. under vacuum, e.g., via vacuum rotary drying, thereby obtaining Form V.

In some embodiments, drying time may range from 5 minutes to 2 hours. In some embodiments, drying time is 5 minutes. In some embodiments, drying time is 2 hours.

The features of each embodiment for above methods of preparing the crystalline forms of compound of Formula A or its solvates can be arbitrary combined. Each embodiment obtained from such arbitrary combinations is included within the scope of the present invention, as if these embodiments obtained from such arbitrary combinations are specifically and individually listed herein.

Pharmaceutical Compositions and Methods of Treatment Compound of Formula A or its solvates, or the crystalline forms of compound of Formula A or its solvates (such as Forms I, Form IV, and Form V) are useful in the treatment of diseases, such as cancer. The cancer includes both primary and metastatic cancers. The cancer includes but not limited to lung cancer (including non-small cell lung cancer, non-small cell lung cancer with brain metastasis), head and neck cancer, (large) intestinal cancer, colorectal cancer, rectal cancer, colon cancer, pancreatic cancer, brain cancer (including glioblastoma), breast cancer, pharynx cancer, epidermoid cancer, ovarian cancer, prostate cancer, gastric cancer, renal cancer, liver cancer, esophageal cancer, bone cancer, sarcoma such as soft tissue sarcoma, and leukemia.

The present invention provides the method of treating cancer responsive to inhibition of overexpression and/or overactivity of epidermal growth factor receptor, comprises administering the active pharmaceutical ingredients consisting of compound of Formula A, or one or more of compound of Formula A or its solvates or the crystalline forms of compound of Formula A or its solvates, such as Forms I, Form IV, or Form V.

In some embodiments, the treatment method is directed to at least one disease responsive to inhibition of overexpression and/or overactivity of epidermal growth factor receptor, such as cancer. An effective amount of a pharmaceutical composition of the present invention is administered to a subject in need thereof, wherein the pharmaceutical composition comprises at least one pharmaceutically acceptable carrier and one or more of compound of Formula A or its solvates or the crystalline forms of compound of Formula A or its solvates (such as Form I, Form IV, or Form V).

The dosing amount of the at least one active pharmaceutical ingredient selected from compound of Formula A, or the crystalline forms of compound of Formula A. or the solvates of compound of Formula A or the crystalline forms thereof (such as Form I, Form IV, or Form V) to achieve the desired biological effect may depend on a number of factors, e.g., the intended use, the mode of administration, and the clinical condition of the patient. The daily dose may, for example, range from 0.01 mg to 3 g/day (such as from 0.05 mg to 2 g/day, even from 100 mg to 1 g/day). Unit dose formulations which can be administered orally include, for example, tablets or capsules.

For the therapy of the above-mentioned conditions, the at least one active pharmaceutical ingredient selected from compound of Formula A or its solvates or the crystalline forms of compound of Formula A or its solvates may be administered as such, but typically in the form of a pharmaceutical composition formulated with one or more pharmaceutically acceptable carriers or excipients.

Representative carriers or excipients should be compatible with the other ingredients of the composition and do not have harmful effect on the patient's health. The carrier or excipient may be a solid or a liquid or both, and may be formulated with compound of Formula A or its solvates or the crystalline forms of compound of Formula A or its solvates (such as Form 1, Form IV, and/or Form V) into a pharmaceutical composition or a unit dosage form (for example, a tablet, a capsule), which may contain from 0.05% to 95% by weight of compound of Formula A.

The pharmaceutical compositions described herein can be produced by known pharmaceutical methods, such as those involving mixing with pharmaceutically acceptable carriers and/or excipients and diluents.

In some embodiments, the at least one active pharmaceutical ingredient selected from compound of Formula A or its solvates or the crystalline forms of compound of Formula A or its solvates (such as Form I, Form IV, and Form V) may be combined with at least one component, such as carrier and/or excipient and/or diluent, which may be selected from sweeteners, flavoring agents, coloring agents, dyes, and emulsifiers.

In some embodiments, the conversion of compound of Formula A or its solvates or the crystalline forms of compound of Formula A or its solvates (such as Form I, Form IV, and Form V) will not occur when formulating with the one or more pharmaceutically acceptable carriers and/or excipients and/or diluents. In other embodiments, compound of Formula A or its solvates or the crystalline forms of compound of Formula A or its solvates (such as Form I, Form IV, or Form V) may be converted, in whole or in part, to one or more other forms, including a non-solid form, when formulating with the one or more pharmaceutically acceptable carriers and/or excipients and/or diluents. In some embodiments, Form I or other forms described herein can be dissolved when formulated into a pharmaceutical composition. Accordingly, in such “dissolved” cases, Form I or other forms no longer exists in their respective crystalline forms in the pharmaceutical composition.

In some embodiments, the at least one active pharmaceutical ingredient selected from compound of Formula A or its solvates or the crystalline forms of compound of Formula A or its solvates (such as Form I, Form IV, and Form V) is formulated into a suitable dosage form.

Pharmaceutical compositions described herein may be dosage forms suitable for oral and peroral (for example sublingual) administration. The suitable mode of administration may depend on not only the condition in each individual case and severity of the condition to be treated, but also the nature of the specific forms of the active pharmaceutical ingredient selected from compound of Formula A or its solvates or the crystalline forms of compound of Formula A or its solvates (such as Form I, Form IV, and Form V) used in preparing the pharmaceutical composition.

Suitable pharmaceutical compositions for oral administration prepared from the at least one active pharmaceutical ingredient selected from compound of Formula A or its solvates or the crystalline forms of compound of Formula A or its solvates (such as Form I, Form IV, and Form V) may be in the form of unit dosage forms such as capsules, cachets, and tablets, including suckable tablets, each of which is prepared with a defined amount of the at least one active pharmaceutical ingredient described herein; as well as in the forms selected from powders, granules, solutions, suspensions in an aqueous or nonaqueous liquid, and oil-in-water and water-in-oil emulsions. Those compositions may, as already mentioned, be prepared by any suitable pharmaceutical formulation methods, such as those including a step wherein the at least one active pharmaceutical ingredient selected from compound of Formula A or its solvates or the crystalline forms of compound of Formula A or its solvates (such as Form I, Form IV, and Form V) and a carrier and/or excipient and/or diluent (which may consist of one or more added ingredients) are brought into contact. The compositions can generally be produced by uniformly and homogeneously mixing the at least one active pharmaceutical ingredient selected from compound of Formula A or its solvates or the crystalline forms of compound of Formula A or its solvates (such as Form I, Form IV, and Form V) with liquid or finely divided solid carriers, after which the product can be shaped.

The at least one active pharmaceutical ingredient selected from compound of Formula A or its solvates or the crystalline forms of compound of Formula A or its solvates (such as Form I, Form IV, and Form V) can also be administered in combination with one or more other active ingredients (such as in the synergetic therapy). When administered as a combination, the active ingredients can be formulated as separate compositions that are administered at the same time or sequentially at different times (such as administered sequentially in any orders) through the same or different administration routes, or the active ingredients can be administered in the same pharmaceutical composition.

In some embodiments, the at least one active pharmaceutical ingredient selected from compound of Formula A or its solvates or the crystalline forms of compound of Formula A or its solvates (such as Form I, Form IV, and Form V) can be administered in combination with one or more other active ingredients with known therapeutical effect, for example for the treatment of diseases responsive to inhibition of overexpression and/or overactivity of epidermal growth factor receptor, such as cancer.

The phrase “combination”, as described herein, defines the combined use of the at least one active pharmaceutical ingredient selected from compound of Formula A or its solvates or the crystalline forms of compound of Formula A or its solvates (such as Form I, Form IV, and Form V) with one or more other active ingredients, such as, the combined use in the anti-neoplastic method. As used herein, the term “anti-neoplastic method” refers to any method for purposes of treating the cancer. Examples of anti-neoplastic method include but not limited to: radiotherapy, immunotherapy, DNA damaging chemotherapy, and chemotherapy that disrupts cell replication.

DNA damaging chemotherapeutic agents include but not limited to, for example, topoisomerase I inhibitors (e.g., irinotecan, topotecan, camptothecin and analogs or metabolites thereof, and adriacin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, dacarbazine, methotrexate, mitomycin, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitabine, gemcitabine, fludarabine, cytarabine, mercaptopurine, thioguanine, pentostatin, and hydroxyurca).

Chemotherapeutic agents those disrupt cell replication include but not limited to: paclitaxel, docetaxel, and related analogs; vincristine, vinblastin, and related analogs; thalidomide and related analogs (e.g., CC-5013 and CC-4047); protein tyrosine kinase inhibitors (e.g., imatinib mesylate, fruquintinib, savolitinib, and gefitinib); proteasome inhibitors (e.g., bortezomib); NF-kappa B inhibitors, including inhibitors of I kappa B kinase; antibodies which bind to proteins overexpressed in cancers and thereby down-regulate cell replication (e.g., rituximab, cetuximab, and bevacizumab); and other inhibitors of proteins or enzymes known to be upregulated, over-expressed, or activated in cancers, the inhibition of which can down-regulates cell replication.

Thus, methods described herein are not limited by the sequence of administration; the one or more other active ingredients may be administered simultaneously to, prior to or after the administration of the at least one active pharmaceutical ingredient. The at least one active pharmaceutical ingredient in the combination described above is selected from compound of Formula A or its solvates or the crystalline forms of compound of Formula A or its solvates (such as Form I, Form IV, and Form V).

The following non-limiting examples are provided.

### EXPERIMENTS

The compound 4-ethyl-N-(4-((3-ethynylphenyl)amino)-7-methoxyquinazolin-6-yl) piperazine-1-carboxamide raw material used in the examples were prepared according to CN101619043A.

All reagents, except intermediates, used in this disclosure are commercially available. The names of all compounds, except the reagents, were generated by ChemBioDraw Ultra 16.0.

Unless otherwise indicated, X-ray powder diffractograms were obtained using Germany Bruker D8 ADVANCE X-ray diffractometer (target: Cu; voltage: 40 kV; electric current: 40 mA; scanning speed: 4 degrees/min; step size: 0.02°; scanning range: 3°-45°).

Differential scanning calorimetry were performed on Perkin Elmer DSC7 (purge gas: nitrogen; flow rate: 50 mL min⁻; heating rate: 5-10° C./min; temperature ranging: 25° C. to 200° C.). The samples were measured in the pricked aluminum pans. Indium was used for temperature calibration.

Thermogravimetric (TG) analysis were obtained using Perkin Elmer TGA7 (purge gas: nitrogen; flow rate: 50 mL min⁻¹; heating rate: 10° C./min).

### Example 1 Preparation of Form I of Compound of Formula A

The 4-ethyl-N-(4-((3-ethynylphenyl)amino)-7-methoxyquinazolin-6-yl)piperazine-1-carboxamide (60 g, 0.139 mol) was dissolved in 150 times (volume/weight ratio) of tetrahydrofuran (9 L) under refluxing. Then the obtained solution was cooled to 50° C., and succinic acid (65.8 g, 0.557 mol, 4 equivalents) was added in one portion. Then the obtained mixed solution was cooled naturally under stirring. The white precipitate was appeared at about 28° C. After further stirring for 18 hours, the white solid was collected by filtration, and dried at 40° C. under vacuum. A powder sample of 56.7 g was obtained (yield 83%).

¹H NMR (400 MHz, cd3od) δ 8.52 (s, 1H), 8.45 (s, 1H), 7.93-7.89 (m, 1H), 7.77-7.73 (m, 1H), 7.35 (t, J=7.9 Hz, 1H), 7.24 (dd, J=5.2, 3.8 Hz, 1H), 7.19 (s, 1H), 4.05 (s, 3H), 3.69-3.61 (m, 4H), 3.49 (s, 1H), 2.71-2.64 (m, 4H), 2.60 (q, J=7.2 Hz, 2H), 2.53 (s, 2H), 1.18 (t, J=7.2 Hz, 3H).

The obtained powder sample is Form I of compound of Formula A, the X-ray powder diffractogram of which is shown in FIG. 1. Peaks (2θ) chosen from the figure has the following values: 6.1, 7.9, 10.2, 11.6, 12.2, 13.6, 15.3, 15.9, 16.6, 17.8, 19.6, 20.4, 21.4, 21.7, 22.3, 23.5, 24.3, and 25.1 degrees, the measured 2θ values each having an error of about ±0.2 degrees (2θ), wherein characteristic peaks (2θ) are at 6.1, 7.9, 12.2, 15.3, 15.9, 16.6, and 20.4 degrees. DSC result is given in FIG. 2, showing that the melting point range of Form I is about 193.4-197.3° C.

### Example 2 Preparation of Form I of Compound of Formula A

Succinic acid (3.5 g) was dissolved in a mixed solvent of i-propanol (50 mL) and H₂O (6 mL) under stirring. Then 4-ethyl-N-(4-((3-ethynylphenyl)amino)-7-methoxyquinazolin-6-yl) piperazine-1-carboxamide (5 g) was added and dissolved under heating and stirring. The solution was filtered. Then additional i-propanol (100 mL) was added. The obtained mixture was stirred for 0.5 hours, then cooled to room temperature and stirred overnight. The solid was collected by filtration and dried to yield 5.45 g of solid product (yield 95.8%). Upon measurement, the X-ray powder diffractogram of the obtained sample is consistent with that of Form I of compound of Formula A obtained in Example 1.

### Example 3 Preparation of Form I of Compound of Formula A

The 4-ethyl-N-(4-((3-ethynylphenyl)amino)-7-methoxyquinazolin-6-yl)piperazine-1-carboxamide (2.15 g, 5 mmol) was dissolved in 21.5 mL of mixed solvent of methanol and dichloromethane (3:9) under stirring. Succinic acid (0.59 g, 5 mmol) was dissolved in 3 mL methanol, and then this acid solution was added into the above free base solution in one portion. Then 32 mL ethyl acetate was added into the mixed solution under stirring. After stirring for 5 hours, the white precipitate was collected by filtration and dried to yield 2.24 g of solid (yield 91.6%). Upon measurement, the X-ray powder diffractogram of the obtained sample is consistent with that of Form I of compound of Formula A obtained in Example 1.

### Example 4 Preparation of Form I of Compound of Formula A

The 4-ethyl-N-(4-((3-ethynylphenyl)amino)-7-methoxyquinazolin-6-yl)piperazine-1-carboxamide (1.3 g) was added into a mixed solvent of ethanol (13 mL) and H₂O (1 mL) and heated to 80° C. for dissolution. To the solution was added succinic acid (0.9 g). The obtained solution was stirred for another 1 hour at 80° C., and then cooled to room temperature under stirring. The precipitate was collected by filtration and dried to yield 0.95 g of solid product (yield 64.3%). Upon measurement, the X-ray powder diffractogram of the obtained sample is consistent with that of Form I of compound of Formula A obtained in Example 1.

### Example 5 Preparation of Form I of Compound of Formula A

The 4-ethyl-N-(4-((3-ethynylphenyl)amino)-7-methoxyquinazolin-6-yl)piperazine-1-carboxamide (1.3 g) was mixed with ethanol (20 mL), and heated to reflux. Then to the mixture was added succinic acid (0.9 g) and the stirring was continued for 2 hours under refluxing. Then the reaction was cooled to room temperature under stirring. The precipitate was collected by filtration and dried to yield 1.23 g of solid product (yield 83.1%). Upon measurement, the X-ray powder diffractogram of the obtained sample is consistent with that of Form I of compound of Formula A obtained in Example 1.

### Example 6 Preparation of Form I of Compound of Formula A

Succinic acid (1.4 g) was dissolved in a mixed solvent of acetone (50 mL) and H₂O (15 mL). Then 4-ethyl-N-(4-((3-ethynylphenyl)amino)-7-methoxyquinazolin-6-yl)piperazine-1-carboxamide (2 g) was added to the solution and stirred to dissolve. The solution was filtered, and then acetone (50 mL) was added to the obtained clear solution. The stirring was continued for 2 hours. The precipitate was collected by filtration and dried under vacuum to yield 1.2 g of solid product (yield 52.6%). Upon measurement, the X-ray powder diffractogram of the obtained sample is consistent with that of Form I of compound of Formula A obtained in Example 1.

### Example 7 Preparation of Form IV of Compound of Formula A

Succinic acid (7.01 g) was dissolved in a mixed solvent of acetonitrile (60 mL) and H₂O (30 mL). Then 4-ethyl-N-(4-((3-ethynylphenyl)amino)-7-methoxyquinazolin-6-yl)piperazine-1-carboxamide (10.01 g) was added slowly and stirred to obtain a clear solution. Then acetonitrile (240 mL) was added, and stirred at room temperature overnight. The precipitate was collected by filtration and dried at room temperature under vacuum for 4 hours. A solid sample of 11.6 g was obtained, yield 83.3%.

¹H NMR (399 MHz, cd3od) δ 8.43 (d, J=1.5 Hz, 1H), 8.40 (t, J=1.3 Hz, 1H), 7.85 (s, 1H), 7.74-7.65 (m, 1H), 7.32 (q, J=7.8 Hz, 1H), 7.22 (dt, J=7.6, 1.2 Hz, 1H), 7.06 (d, J=2.0 Hz, 1H), 3.97 (d, J=0.9 Hz, 3H), 3.73-3.63 (m, 4H), 3.52 (d, J=1.7 Hz, 1H), 2.86-2.79 (m, 4H), 2.75 (q, J=7.3 Hz, 2H), 2.53 (s, 4H), 1.22 (t, J=7.3 Hz, 3H).

The obtained powder sample is Form IV of compound of Formula A, the X-ray powder diffractogram of which is shown in FIG. 4. Peaks (2θ) chosen from the figure has the following values: 5.4, 7.3, 8.5, 9.6, 11.9, 12.2, 13.7, 14.5, 15.3, 17.5, 18.5, 19.1, 20.6, 20.9, 21.9, 22.8, 24.1, 26.6, 27.1, 27.4, and 27.8 degrees, the measured 2θ values each having an error of about ±0.2 degrees (2θ), wherein characteristic peaks (2θ) are at 5.4, 8.5, 12.2, 14.5, and 15.3 degrees. DSC result is given in FIG. 5, showing that the melting point range of Form IV is about 70.6-81.4° C.

### Example 8 Preparation of Form IV of Compound of Formula A

Succinic acid (0.55 g) was dissolved in a mixed solvent of acetonitrile (6 mL) and H₂O (3 mL). Then 4-ethyl-N-(4-((3-ethynylphenyl)amino)-7-methoxyquinazolin-6-yl)piperazine-1-carboxamide (1 g) was added slowly and stirred to obtain a clear solution. Then acetonitrile (24 mL) was added and stirred at room temperature overnight. The precipitate was collected by filtration and dried at room temperature under vacuum for 4 hours to yield 1.1 g of solid sample (yield 82%). Upon measurement, the X-ray powder diffractogram of the obtained sample is consistent with that of Form IV of compound of Formula A obtained in Example 7.

### Example 9 Preparation of Form V of Compound of Formula A

The sample of Form IV of compound of Formula A (150 mg) was dried at 60° C. via vacuum rotary drying for 5 minutes. A solid sample of 130 mg was obtained, yield 86.6%.

¹H NMR (399 MHz, cd3od) δ 8.53-8.47 (m, 1H), 8.44 (d, J=1.7 Hz, 1H), 7.90 (t, J=1.8 Hz, 1H), 7.74 (ddd, J=8.2, 2.2, 1.1 Hz, 1H), 7.39-7.30 (m, 1H), 7.24 (dt, J=7.6, 1.3 Hz, 1H), 7.16 (d, J=1.9 Hz, 1H), 4.03 (s, 3H), 3.72-3.64 (m, 4H), 3.51 (d, J=1.7 Hz, 1H), 2.79-2.73 (m, 4H), 2.73-2.64 (m, 2H), 2.53 (s, 4H), 1.21 (t, J=7.3 Hz, 3H).

The obtained powder sample is Form V of compound of Formula A, the X-ray powder diffractogram of which is shown in FIG. 7. Peaks (2θ) chosen from the figure has the following values: 6.0, 8.9, 9.6, 12.0, 14.5, 15.6, 16.2, 17.5, 19.2, 20.2, 22.5, 23.9, 27.2, and 27.8 degrees, the measured 2θ values each having an error of about ±0.2 degrees (2θ), wherein characteristic peaks (2θ) are at 6.0, 8.9, 9.6, and 14.5 degrees. DSC result is given in FIG. 8, showing that the endothermic peaks of Form V are at about 77.4-85.8° C., about 147.0-153.9° C., and about 165.1-173.4° C.

### Example 10 Preparation of Form V of Compound of Formula A

The sample of Form IV of compound of Formula A (200 mg) was dried at 60° C. via vacuum rotary drying for 2 hours. A solid sample of 175 mg was obtained, yield 87.5%. Upon measurement, the X-ray powder diffractogram of the obtained sample is consistent with that of Form V of compound of Formula A obtained in Example 9.

### Example 11 Stability of Form I Under High Temperature, High Humidity and Illumination Conditions

Determination method: the test sample of Form I of compound of Formula A was placed on a culture dish, which was uncovered and placed in sealed clean containers. The containers were placed under a temperature of 60° C. and an illumination of 4500 lx±500 lx respectively for 10 days; or the containers were placed under a temperature of 25° C. and a relative humidity of 92.5%%±5% and under a temperature of 40° C. and a relative humidity of 75%±5% respectively for 2 weeks.

Then sampled and investigated for the purity (using HPLC analysis) and crystalline form (using X-ray powder diffraction analysis) of the sample. The investigation results were compared and shown in Table 1 and Table 2.

### Table 1 Results of Stability Experiments of Form I Under High Temperature and Illumination Conditions (10 Days)

| | 0 day | | 60° C. | | 4500 lx ± 500 lx | |
|---|---|---|---|---|---|---|
| Form | Purity A % | Form | Purity A % | Form | Purity A % | Form |
| I | 99.37 | I | 98.68 | I | 99.29 | I |

### Table 2 Results of Stability Experiments of Form I Under High Humidity Condition 2 Weeks

| | 0 day | | 92.5 ± 5% RH/25° C. | | 75 ± 5% RH/40° C. | |
|---|---|---|---|---|---|---|
| Form | Purity A % | Form | Purity A % | Form | Purity A % | Form |
| I | 98.98 | I | 98.98 | I | 99.08 | I |

Conclusion: the data in Table 1 and Table 2 illustrate that, the chemical purity and crystalline form of Form I are not changed after placed under high temperature and illumination conditions for 10 days, and after placed under high humidity condition for 2 weeks, showing that Form I is stable.

### Example 12 Hygroscopicity of Form I

Determination method: the test sample of Form I was weighed and placed on a culture dish, which was uncovered and placed in a sealed clean container with the relative humidity of 92.5%. Then the container was placed at room temperature for 10 days. Then sampled, weighed the sample after placed for 10 days, and compared with the weight of the sample before the test. Thereby the percentage of weight gain by hygroscopicity of the sample was calculated. The crystalline form was also investigated (using X-ray powder diffraction analysis). The results were shown in Table 3.

TABLE 3

| | |
|---|---|
| RH (%) | 92.5% RH, 25° C. |
| Weight gain after 10 days (%) | 0.01% |
| Crystal form after 10 days | I |

Conclusion: the data in the table illustrate that, the weight gain by hygroscopicity of Form I is only 0.01% after placed under high humidity condition for 10 days, i.e., Form I is non-hygroscopic; the crystalline form is not changed during the placement, i.e., Form I is stable.

### Example 13 Solubility Test of Form I

Determination method: excess amount of Form I sample of compound of Formula A and its free base (compound 4-ethyl-N-(4-((3-ethynylphenyl)amino)-7-methoxyquinazolin-6-yl) piperazine-1-carboxamide) were suspended in different dissolution media respectively, and saturated for 24 hours under shaking at constant temperature, then filtered. The filtrates were used for determining solubility of the samples. The results were shown in Table 4. The buffers of different pH, simulated gastric fluid, and simulated intestinal fluid were prepared according to the US pharmacopeia (USP40-NF35).

TABLE 4

| | Solubility | | Solubility | | Test temperature |
|---|---|---|---|---|---|
| Dissolution Media | (Form I) | | (free base) | | (° C.) |
| pH 1.2 buffer | 103.2 | mg/ml | 38.5 | mg/ml | 25° C. |
| pH 2.1 buffer | 15.5 | mg/ml | 5.45 | mg/ml | |
| pH 4.5 buffer | 10.0 | mg/ml | 5.21 | μg/ml | |
| pH 6.8 buffer | 253.1 | μg/ml | 0.84 | μg/ml | |
| pH 7.4 buffer | 46.1 | μg/ml | 0.41 | μg/ml | |
| pH 8.0 buffer | 0.5 | μg/ml | 0.0 | μg/ml | |
| Simulated gastric | 103.0 | mg/ml | 38.5 | mg/ml | 20° C. |
| fluid (SGF) | | | | | |
| Simulated intestinal | 253.0 | μg/ml | 0.84 | μg/ml | |
| fluid (SIF) | | | | | |
| Distilled water | 803.0 | μg/ml | 1.0 | μg/ml | |

Conclusion: the data in the table illustrate that, the solubility of compound of Formula A is significantly higher than that of its free base.

It is to be understood that, the examples and embodiments described herein are only for interpretation purposes, and various improvements or modifications in view of these would be suggested to those skilled in the art and are within the spirit and scope of present application and the scope of the appended claims. All the publications, patents and patent applications cited herein are incorporated herein by reference for all purposes.

1. The succinate salts of 4-ethyl-N-(4-((3-ethynylphenyl)amino)-7-methoxyquinazolin-6-yl)piperazine-1-carboxamide.

2. The succinate salts of 4-ethyl-N-(4-((3-ethynylphenyl)amino)-7-methoxyquinazolin-6-yl)piperazine-1-carboxamide according to claim 1, represented by Formula A: 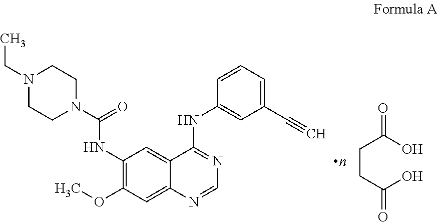
wherein, n is 0.5 or 1.

3. The salt according to claim 2, wherein n is 0.5.

4. The salt according to claim 2, wherein n is 1.

5. The salt according to claim 3, being Form I, characterized in that the X-ray powder diffractogram of Form I includes characteristic peaks at the following 2-theta values: 6.1, 7.9, 12.2, 15.3, 15.9, 16.6, and 20.4 degrees, the measured 2θ values each having an error of about ±0.2 degrees (2θ).

6. The salt according to claim 5, being Form I, characterized in that the X-ray powder diffractogram of Form I includes characteristic peaks at the following 2-theta values: 6.1, 7.9, 10.2, 11.6, 12.2, 15.3, 15.9, 16.6, 17.8, 19.6, 20.4, 21.7, and 23.5 degrees, the measured 2θ values each having an error of about ±0.2 degrees (2θ).

7. A pharmaceutical composition, characterized in that it comprises an effective amount of one or more salts according to claim 2, and a pharmaceutically acceptable carrier.

8. (canceled)

9. A method of treating diseases associated with overexpression and/or overactivity of EGFR, such as cancer, comprising administering to a subject in need thereof an effective amount of a salt according to claim 2, wherein said cancer is selected from lung cancer (including non-small cell lung cancer, non-small cell lung cancer with brain metastasis), head and neck cancer, (large) intestinal cancer, colorectal cancer, rectal cancer, colon cancer, pancreatic cancer, brain cancer, breast cancer, pharynx cancer, epidermoid cancer, ovarian cancer, prostate cancer, gastric cancer, renal cancer, liver cancer, esophageal cancer, bone cancer, sarcoma such as soft tissue sarcoma, and leukemia.

10. (canceled)

11. A method of preparing a salt according to claim 5, said salt being Form I, comprising:
(1) mixing the compound 4-ethyl-N-(4-((3-ethynylphenyl)amino)-7-methoxyquinazolin-6-yl)piperazine-1-carboxamide with succinic acid in an appropriate amount of at least one dissolution solvent (such as C₁₋₆ alkanol, tetrahydrofuran, haloalkane with less than three carbon atoms, acetone, butanone, or acetonitrile) or of a mixed solvent consisting of water miscible organic solvent (such as acetone, methanol, ethanol, i-propanol, tetrahydrofuran, or acetonitrile) and water, heating and stirring to form a salt;
(2) naturally cooling the reaction solution obtained in step (1) to room temperature with stirring;
(3) isolating to obtain the solid Form I of 4-ethyl-N-(4-((3-ethynylphenyl)amino)-7-methoxyquinazolin-6-yl)piperazine-1-carboxamide succinate;
(4) drying the solid obtained in step (3).

12. A method of preparing a salt according to claim 5, said salt being Form I, comprising:
(1) adding the compound 4-ethyl-N-(4-((3-ethynylphenyl)amino)-7-methoxyquinazolin-6-yl) piperazine-1-carboxamide and succinic acid into an appropriate amount of at least one dissolution solvent (such as a mixed solvent of methanol and dichloromethane) or of a mixed solvent consisting of water miscible organic solvent (such as acetone or i-propanol) and water, and reacting to form a salt, thereby obtaining the first solution;
(2) adding at least one anti-dissolution solvent (such as ethyl acetate, acetone, or i-propanol) into said first solution to obtain the second solution;
(3) isolating to obtain the solid Form I of 4-ethyl-N-(4-((3-ethynylphenyl)amino)-7-methoxyquinazolin-6-yl)piperazine-1-carboxamide succinate;
(4) drying the solid obtained in step (3).

13. A pharmaceutical composition, characterized in that it comprises a salt according to claim 5, and a pharmaceutically acceptable carrier; wherein the content of other crystalline forms of the salt in said pharmaceutical composition is less than 40% by weight, based on the total weight of the forms.

14. The pharmaceutical composition according to claim 13, wherein the content of other crystalline forms of the salt in said pharmaceutical composition is less than 30% by weight, based on the total weight of the forms.

15. The pharmaceutical composition according to claim 13, wherein the content of other crystalline forms of the salt in said pharmaceutical composition is less than 20% by weight, based on the total weight of the forms.

16. The pharmaceutical composition according to claim 13, wherein the content of other crystalline forms of the salt in said pharmaceutical composition is less than 10% by weight, based on the total weight of the forms.

17. The pharmaceutical composition according to claim 13, wherein the content of other crystalline forms of the salt in said pharmaceutical composition is less than 5% by weight, based on the total weight of the forms.

18. The pharmaceutical composition according to claim 13, wherein the content of other crystalline forms of the salt in said pharmaceutical composition is less than 1% by weight, based on the total weight of the forms.

19. A pharmaceutical composition, characterized in that it comprises a salt according to claim 5, a pharmaceutically acceptable carrier, and one or more other therapeutically active compounds; wherein the content of other crystalline forms of the salt in said pharmaceutical composition is less than 40% by weight, based on the total weight of the forms.

20. A method of preparing a salt according to claim 6, said salt being Form I, comprising:
(1) mixing the compound 4-ethyl-N-(4-((3-ethynylphenyl)amino)-7-methoxyquinazolin-6-yl)piperazine-1-carboxamide with succinic acid in an appropriate amount of at least one dissolution solvent (such as C₁₋₆ alkanol, tetrahydrofuran, haloalkane with less than three carbon atoms, acetone, butanone, or acetonitrile) or of a mixed solvent consisting of water miscible organic solvent (such as acetone, methanol, ethanol, i-propanol, tetrahydrofuran, or acetonitrile) and water, heating and stirring to form a salt;
(2) naturally cooling the reaction solution obtained in step (1) to room temperature with stirring;
(3) isolating to obtain the solid Form I of 4-ethyl-N-(4-((3-ethynylphenyl)amino)-7-methoxyquinazolin-6-yl)piperazine-1-carboxamide succinate;
(4) drying the solid obtained in step (3).

21. A method of preparing a salt according to claim 6, said salt being Form I, comprising:
(1) adding the compound 4-ethyl-N-(4-((3-ethynylphenyl)amino)-7-methoxyquinazolin-6-yl) piperazine-1-carboxamide and succinic acid into an appropriate amount of at least one dissolution solvent (such as a mixed solvent of methanol and dichloromethane) or of a mixed solvent consisting of water miscible organic solvent (such as acetone or i-propanol) and water, and reacting to form a salt, thereby obtaining the first solution;
(2) adding at least one anti-dissolution solvent (such as ethyl acetate, acetone, or i-propanol) into said first solution to obtain the second solution;
(3) isolating to obtain the solid Form I of 4-ethyl-N-(4-((3-ethynylphenyl)amino)-7-methoxyquinazolin-6-yl)piperazine-1-carboxamide succinate;
(4) drying the solid obtained in step (3).
